(19) United States
(12) Patent Application Publication
(10) Pub. No.: US 2010/0234715 A1
(43) Pub. Date: Sep. 16, 2010

(54) GARMENT FOR MEASURING PHYSIOLOGICAL SIGNALS AND METHOD OF FABRICATING THE SAME

(75) Inventors: Seung-Chul Shin, Daejeon (KR); Yong-Won Jang, Daejeon (KR); In-Bum Lee, Daejeon (KR); Seung-Hwan Kim, Daejeon (KR); Seon-Hee Park, Daejeon (KR)

Correspondence Address: AMPACC Law Group, 3500 188th Street S.W., Suite 103, Lynnwood, WA, 98037, US

(73) Assignee: Electronics and Telecommunications Research Institute, Daejeon (KR)

(21) Appl. No.: 12/671,506

(22) PCT Filed: May 8, 2008
(86) PCT No.: PCT/KR08/02593

§ 371 Date: Jan. 29, 2010

(30) Foreign Application Priority Data
Aug. 3, 2007 (KR) 10-2007-0078257

(51) Int. Cl. A61B 5/04; D05B 23/00; D04B 1/24

(52) U.S. Cl. 600/388; 112/475.09; 66/171

(57) ABSTRACT

Provided is a garment for measuring physiological signals. The garment includes at least one electrode sensor, a signal connection line, a snap structure, and a measurement unit. The electrode sensor is coupled to an inner surface of the garment to make contact with a skin for detecting physiological signals. The signal connection line transmits the physiological signals detected by the electrode sensor. The signal connection line is finished against the inner surface of the garment. The snap structure is coupled to a portion of the garment where the electrode sensor is not overlapped and is electrically connected to the signal connection line. The measurement unit is mounted on the snap structure for measuring the physiological signals. The signal connection line has elasticity.

### TECHNICAL FIELD

The present invention disclosed herein relates to a garment for measuring physiological signals and a method of fabricating the garment, and more particularly, to a garment for stably measuring physiological signals even when a user moves or takes vigorous exercise, and a method of fabricating the garment.

The present invention has been derived from research undertaken as a part of IT R & D program of the Ministry of Information and Communication and Institution of Information Technology Association (MIC/IITA) [2006-S-007-02], Ubiquitous health monitoring module and system development.

### BACKGROUND ART

Recent attempts for ubiquitous-healthcare include attaching sensors to a garment of a person to obtain information about health conditions of the person. The sensors should be securely kept in contact with a skin of the person to measure physiological signals for obtaining information about the health conditions of the person, such as electrocardiograms, pulse rates, respiratory rates, body fat, and obesity.

To measure an electrocardiogram or respiratory rate, a sensor must be steadily kept in contact with the skin of the person for a long time. In addition, it is necessary to measure the physiological signals accurately and make the person feel comfortable during the measurement. For this, a garment can be made of an elastic material such as spandex yarn, and electrode sensors that make contact with the skin can be made of a material having garment-like elasticity.

When a measurement unit and an electrode sensor are distantly attached to the garment made of the elastic material such as spandex yarn, it is important to select a proper signal connection line for connecting the measurement unit and the electrode sensor.

If the signal connection line has not elastic, the garment can be badly deformed due to an inelastic signal connection line when the person with the garment moves or takes exercise. Moreover, distorted signals or noises can be generated.

### DISCLOSURE OF INVENTION

### Technical Problem

An ubiquitous-healthcare garment is needed for stably measuring physiological signals even when a user takes vigorous exercise as well as during everyday life activities of the user.

Also, a method of fabricating an ubiquitous-healthcare garment is needed for stably measuring physiological signals even when a user takes vigorous exercise as well as during everyday life activities of the user.

### Technical Solution

Embodiments of the present invention provide garments for measuring physiological signals, the garments including: an electrode sensor coupled to an inner surface of a garment to make contact with a skin for detecting physiological signals; a signal connection line connected to the electrode sensor; a snap structure electrically connected to the signal connection line; and a measurement unit mounted on the snap structure for measuring the physiological signals, wherein the signal connection line has elasticity.

In some embodiments, a portion of the garment which coupled to the electrode sensor is designed for applying a pressure equal to or higher than 0.1 kPa. The garment may include spandex yarn.

In other embodiments, the electrode sensor is a conductive fabric electrode formed of conductive yarn. The conductive fabric electrode may be formed by a tricot method or a knit method. The conductive fabric electrode may be more elasticity than the garment. The conductive yarn may be a thread of a filament or staple structure coated with a conductive material. The conductive material may include silver (Ag).

In still other embodiments, the garment further includes a coupling adhesive member used to couple the electrode sensor to the inner surface of the garment. The coupling adhesive member may include: a seam sealing or hot-melt tape; and a fabric bonded to the tape, wherein the fabric is the same as that used for forming the garment.

In even other embodiments, the garment further includes an anti-slipping adhesive member provided along a border between the electrode sensor and the coupling adhesive member. The anti-slipping adhesive member may be a hot-melt or silicon-based tape.

In yet other embodiments, the garment further includes an interconnection adhesive member configured to connect the electrode sensor and the signal connection line. The interconnection adhesive member may be more elasticity than the electrode sensor. The interconnection adhesive member may be a seam sealing or hot-melt tape.

In further embodiments, the garment further includes an interconnection metal structure configured to connect the electrode sensor and the signal connection line. The interconnection metal structure may have a yoyo shape in which a pair of circular disks is disposed on both sides of a central post passing through the electrode sensor. The signal connection line may be connected to the electrode sensor by winding a portion of the signal connection line around the central post of the interconnection metal structure.

In still further embodiments, the signal connection line includes: an elastic thread as a core material; a metal thread wound around the elastic thread; and an insulation thread wound around the metal thread to cover the metal thread.

In even further embodiments, the elastic thread includes an elastic material. The metal thread may be coated with a conductive material. The conductive material may include silver (Ag). The insulation thread may include polyester fabric.

In yet further embodiments, the signal connection line is finished against the inner surface of the garment. The garment may further include a finishing adhesive member for finishing the signal connection line. The finishing adhesive member may be more elasticity than the signal connection line. The finishing adhesive member may be a seam sealing or hot-melt tape.

In some embodiments, the snap structure includes: a male snap including a post passing through the garment; and a female snap coupled to the male snap with the garment being disposed between the female snap and the male snap. The signal connection line may have a portion wound around the post of the male snap. The garment may further include a conductive material disposed between the garment and the male snap. The garment may further include a snap structure bonding member bonded to the inner surface of the garment for covering the snap structure.

In other embodiments, the snap structure is coupled to a portion of the garment to which the electrode sensor is not overlapped.

In still other embodiments, the garment further includes a pocket unit disposed on an outer surface of the garment for pocketing the measurement unit.

In other embodiments of the present invention, there are provided methods of fabricating a physiological signal measuring garment, the methods include: coupling an electrode sensor to an inner surface of a garment to allow the electrode sensor to make contact with a skin for detecting physiological signals; connecting a signal connection line to the electrode sensor; forming a snap structure electrically connected to the signal connection line; and mounting a measurement unit on the snap structure, wherein the signal connection line has elasticity.

In some embodiments, a portion of the garment which coupled to the electrode sensor is designed for applying a pressure equal to or higher than 0.1 kPa. The garment may be formed of spandex yarn.

In other embodiments, the electrode sensor is a conductive fabric electrode formed of conductive yarn. The conductive fabric electrode may be formed of conductive yarn by a tricot method or a knit method. The conductive fabric electrode may be more elasticity than the garment. The conductive yarn may be a thread of a filament or staple structure coated with a conductive material. The conductive material may include silver (Ag).

In still other embodiments, the coupling of the electrode sensor to the inner surface of the garment includes: determining a portion of the garment to which the electrode sensor is to be coupled; and coupling the electrode sensor to the determined portion of the garment using a coupling adhesive member. The coupling adhesive member may include: a seam sealing or hot-melt tape; and a fabric bonded to the tape, wherein the fabric is the same as that used for forming the garment.

In even other embodiments, the method further includes forming an anti-slipping adhesive member along a border between the electrode sensor and the coupling adhesive member. The anti-slipping adhesive member may be a hot-melt or silicon-based tape.

In yet other embodiments, the connecting the signal connection line to the electrode sensor may use an interconnection adhesive member.

In further embodiments, the connecting of the signal connection line to the electrode sensor using the interconnection adhesive member includes: placing a portion of the signal connection line on the electrode sensor; and covering the portion of the signal connection line with the interconnection adhesive member. The interconnection adhesive member may be more elasticity than the electrode sensor. The interconnection adhesive member may be a seam sealing or hot-melt tape.

In still further embodiments, the connecting the signal connection line to the electrode sensor may use an interconnection metal structure.

In even further embodiments, the connecting of the signal connection line to the electrode sensor using the interconnection metal structure includes: forming a hole through the electrode sensor; inserting a metal structure having a post corresponding to the hole into the hole; winding a portion of the signal connection line around the post of the metal structure; and deforming the metal structure to form the interconnection metal structure, the metal structure has a yoyo shape in which a pair of circular disks are disposed on both sides of a central post passing through the electrode sensor.

In yet further embodiments, the signal line is formed by a method including: preparing an elastic thread using a core material having an elastic material; winding the elastic thread with a metal thread coated with a conductive material; and winding the metal thread with an insulation thread formed of polyester fabric to cover the metal thread.

In some embodiments, the method further includes finishing the signal connection line against the inner surface of the garment. The finishing of the signal connection line against the inner surface of the garment may include attaching a finishing adhesive member to the inner surface of the garment to cover the signal connection line. The finishing adhesive member may be more elasticity than the signal connection line. The finishing adhesive member may be a seam sealing or hot-melt tape.

In other embodiments, the forming of the snap structure includes: determining a portion of the garment to which the snap structure is coupled; forming a hole through the determined portion of the garment; inserting a male snap having a post corresponding to the hole into the hole; and coupling a female snap to a portion of the post of the male snap protruding from an outer surface of the garment so as to form the snap structure into a yoyo shape in which a pair of circular disks are disposed on both sides of a central post passing through the garment.

In still other embodiments, the method further includes winding a portion of the signal connection line around the post of the mail snap. The method may further include disposing a conductive material between the garment and the male snap. The method may further include finishing the inner surface of the garment with a snap structure bonding member to cover the snap structure.

In even other embodiments, the snap structure is coupled to a portion of the garment to which the electrode sensor is not overlapped.

In yet other embodiments, the method further includes forming a pocket unit on an outer surface of the garment for pocketing the measurement unit.

### ADVANTAGEOUS EFFECTS

As described above, according to the present invention, although the physiological signal measuring garment can be folded and/or stretched when a user takes exercise, distortion and noise of detected physiological signals can be kept below a low level. That is, physiological signals of the user can be stably measured over a long time period during everyday life activities or sport activities of the user, such as running and gymnastics. Therefore, the physiological signal measuring garment is useful for healthcare.

Furthermore, since the electrode sensor of the physiological signal measuring garment can be adjusted according to the kinds of physiological signals to be measured, various physiological signals can be measured, such as 1-chanel or 3-chanel electrocardiogram signals, respiratory waveform signals, and belly or left/right body fat signals. In addition, since the electrode sensors and the measurement unit can be attached to any portions of the physiological signal measuring garment as long as the user does not feel uncomfortable, the physiological signal measuring garment can be flexibly designed.

### BRIEF DESCRIPTION OF THE DRAWINGS

The accompanying figures are included to provide a further understanding of the present invention, and are incorporated in and constitute a part of this specification. The drawings illustrate exemplary embodiments of the present invention and, together with the description, serve to explain principles of the present invention. In the figures:

FIG. 1 is a schematic view illustrating a physiological signal measuring garment according to an embodiment of the present invention;

FIG. 2 is a flowchart for explaining a method of fabricating a physiological signal measuring garment according to an embodiment of the present invention;

FIG. 3 is a perspective view illustrating a signal connection line of a physiological signal measuring garment according to an embodiment of the present invention;

FIG. 4 is a perspective view illustrating an electrode sensor unit of a physiological signal measuring garment according to an embodiment of the present invention;

FIG. 5 is a sectional view taken along line A-A′ of FIG. 4;

FIG. 6 is a plan view illustrating an electrode sensor unit of a physiological signal measuring garment according to another embodiment of the present invention;

FIG. 7 is a sectional view taken along line B-B′ of FIG. 6;

FIGS. 8 and 9 are front and plan views illustrating a metal structure used in the electrode sensor unit of FIG. 6 according to an embodiment of the present invention;

FIGS. 10 and 12 are plan views illustrating coupled electrode sensors and finished signal connection lines to a physiological signal measuring garment according to embodiments of the present invention;

FIGS. 11 and 13 are sectional views taken along lines C-C′ of FIG. 10 and line D-D′ of FIG. 12;

FIG. 14 is a flowchart for explaining a method of coupling an electrode sensor to a physiological signal measuring garment and finishing a signal connection line according to an embodiment of the present invention;

FIG. 15 is a plan view illustrating snap structures coupled to a physiological signal measuring garment according to an embodiment of the present invention;

FIG. 16 is a sectional view taken along line E-E′ of FIG. 15; and

FIG. 17 is a flowchart for explaining a method of forming a snap structure of a physiological signal measuring garment according to an embodiment of the present invention.

### BEST MODE FOR CARRYING OUT THE INVENTION

Preferred embodiments of the present invention will be described below in more detail with reference to the accompanying drawings. The present invention may, however, be embodied in different forms and should not be construed as limited to the embodiments set forth herein. Rather, these embodiments are provided so that this disclosure will be thorough and complete, and will fully convey the scope of the present invention to those skilled in the art. In the figures, the dimensions of layers and regions are exaggerated for clarity of illustration, and like reference numerals refer to like elements throughout.

Hereinafter, an exemplary embodiment of the present invention will be described with the accompanying drawings.

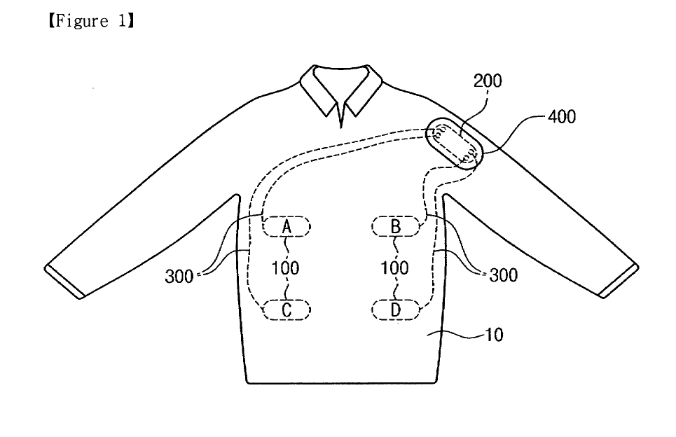

FIG. 1 is a schematic view illustrating a physiological signal measuring garment according to an embodiment of the present invention.

Referring to FIG. 1, the physiological signal measuring garment may include a garment 10, electrode sensor units 100, a snap coupler 200, signal connection lines 300, a measurement unit (not shown), and a pocket unit 400.

The garment 10 may be formed of an elastic material. For example, the garment 10 may be formed of the elastic material such as spandex yarn. Portions of the garment 10 which coupled to the electrode sensor units 100 are designed for applying a pressure higher than about 0.1 kPa in order to tightly push the electrode sensor units 100 to a user's skin.

The electrode sensor units 100 may be brought into tight contact with the user's skin for measuring physiological signals. The electrode sensor units 100 may include fabric electrodes formed of conductive yarn. Conductive fabric electrodes can be formed of conductive yarn by a tricot method or a knit method. In this case, the electrode sensor units 100 may be elastic. The electrode sensor units 100 may be more elasticity than fabric used for making the garment 10. The conductive yarn may be polyester thread having a filament or staple structure coated with conductive material. The conductive material used for coating the polyester thread may include silver (Ag) since silver does not cause skin troubles.

The electrode sensor units 100 may be selectively attached to the garment 10 according to the kinds of physiological signals to be measured. For example, when it is intended to measure 1-chanel electrocardiogram signals, at least one electrode sensor unit 100 may be coupled to portion A and/or B of the garment 10 corresponding to a user's chest. When it is intended to measure 3-chanel electrocardiogram signals, a plurality of electrode sensor units 100 may be coupled to portion A (right arm: RA), portion B (left arm: LA), portion C (right leg: RL), and/or portion D (left leg: LL) of the garment 10. When it is intended to measure respiratory waveform signals, a plurality of electrode sensor units 100 may be coupled to portions A and D, or portions B and C.

When it is intended to measure body fat, at least one electrode sensor unit 100 may be coupled to each of portion A, portion B, portion C, and/or portion D. Here, instead of portions A and B, the electrode sensor unit 100 may be coupled to a portion of the garment 10 corresponding to a user's shoulder or forearm. In this case, information about abdominal fat and/or upper body fat may be detected. The electrode sensor units 100 may be coupled to various portions of the garment 10 including portions A, B, C, and D. In this case, physiological signals containing information about body fat can be detected from various portions the user's body.

The measurement unit may be mounted to the garment 10 using the snap coupler 200. The snap coupler 200 may be coupled to a portion of the garment 10 where the electrode sensor units 100 are not overlapped. In the embodiment of FIG. 1, the snap coupler 200 may be coupled to an upper arm portion of the garment 10. According to the design, convenience, and purpose of the garment 10, the position of the snap coupler 200 may be varied. For example, the snap coupler 200 may be coupled to other portions of the garment 10 where the electrode sensor units 100 are not overlapped, such as upper chest portions, belly portions, shoulder portions, back portions, rib portions, wrist portions, upper arm portions, and forearm portions.

The measurement unit may be a device capable of displaying measurement values by performing operations such as filtering, amplification, and conversion on the physiological signals detected using the electrode sensor units 100. The measurement unit may be mounted to the snap coupler 200. The pocket unit 400 may provide a room for pocketing the measurement unit mounted to the snap coupler 200.

The signal connection lines 300 may be electrically connected to the snap coupler 200 to transmit the physiological signals detected by the electrode sensor units 100 to the measurement unit. The signal connection lines 300 may be elastic. The signal connection lines 300 may be as elastic as fabric used for making the garment 10 or more elasticity than the fabric.

In the current embodiment, the garment 10, the electrode sensor units 100, and the signal connection lines 300 of the physiological signal measuring garment are elastic. Therefore, although the physiological signal measuring garment may be folded and/or stretched when the user moves or takes exercise, distortion and noise of detected physiological signals may be kept below a low level. That is, the physiological signals may be easily detected even when the user moves or takes vigorous exercise. Furthermore, since the electrode sensor units 100 may be coupled to desired portions of the garment 10, various physiological signals may be detected.

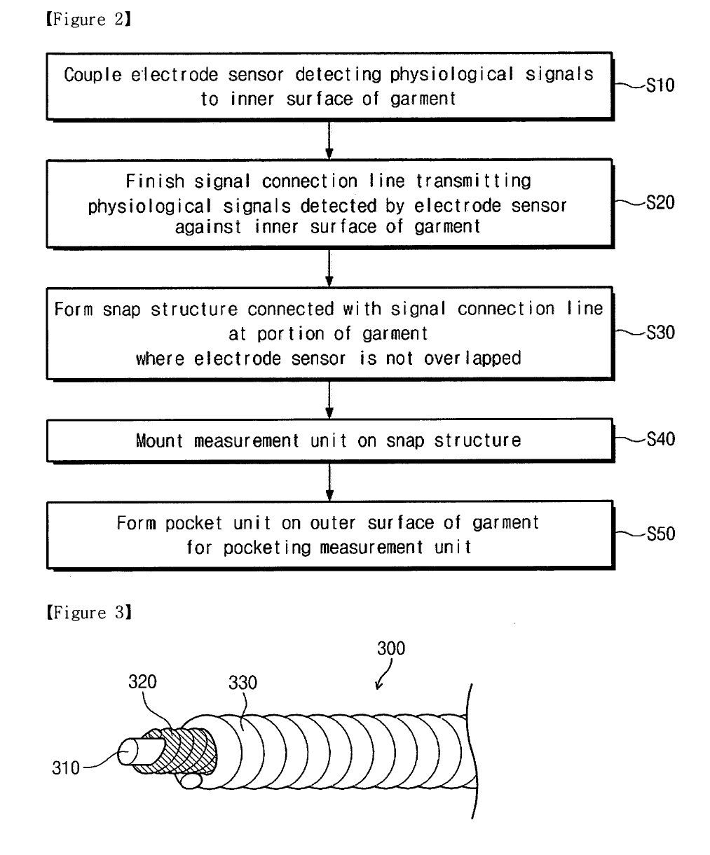

FIG. 2 is a flowchart for explaining a method of fabricating a physiological signal measuring garment according to an embodiment of the present invention.

Referring to FIG. 2, the method may include: operations S10, S20, S30, S40, and S50. In operation S10, an electrode sensor for measuring physiological signals is coupled to an inner surface of a garment. In operation S20, finishing is performed on a signal connection line, which is disposed to the inner surface of the garment for transmitting physiological signals detected by the electrode sensor. In operation S30, a snap structure is formed on a portion of the garment where the electrode sensor is not overlapped and is connected to the signal line. In operation S40, a measurement unit is mounted to the snap structure. In operation S50, a pocket unit is formed on an outer surface of the garment to pocket the measurement unit.

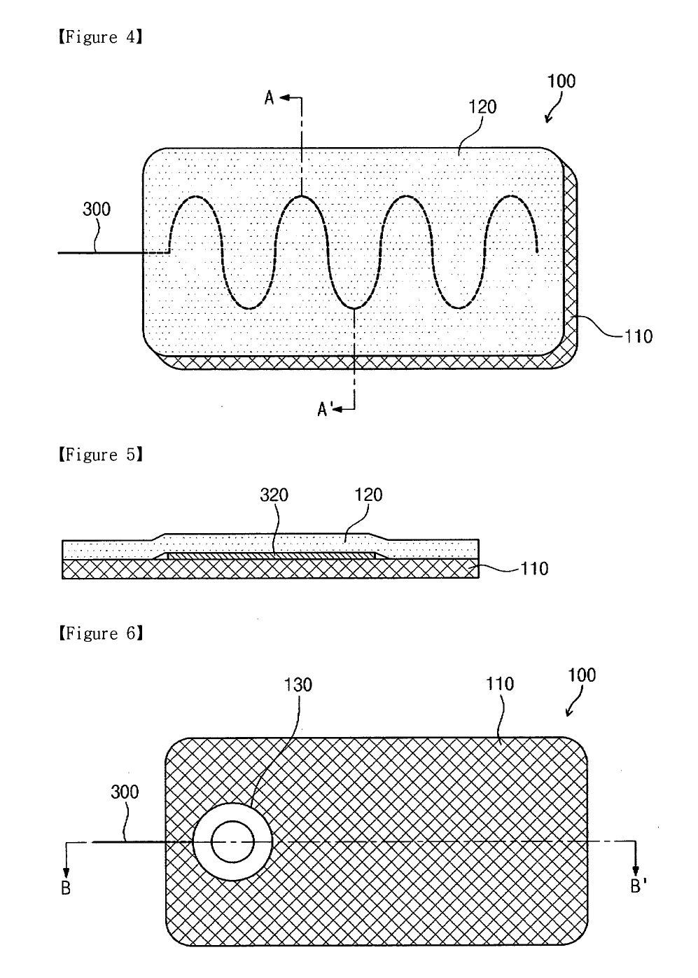

The method of fabricating a physiological signal measuring garment will be described in more detail with reference to FIGS. 4 through 13, and 15 and 16.

In operation S10, an electrode sensor 110 may be coupled to an inner surface of a garment 10. For this, a portion of the garment 10 where the electrode sensor 110 is to be coupled may be first selected, and the electrode sensor 110 may be attached to the selected portion of the garment 10 using a coupling adhesive member 121. The coupling adhesive member 121 may be elastic. The coupling adhesive member 121 may be as elastic as fabric used for making the garment 10 or more elasticity than the fabric. In this case, even when a user takes vigorous exercise, the electrode sensor 110 may be stably positioned on the garment 110 owing to the coupling adhesive member 121. The coupling adhesive member 121 may be formed by bonding a piece of fabric used for making the garment 10 to a seam sealing or hot-melt tape. For example, the coupling adhesive member 121 may be formed by bonding a piece of fabric used for making the garment 10 to a hot-melt tape. In this case, it may be difficult to distinguish the coupling adhesive member 121 from the garment 10, and thus the garment 10 may have a neat appearance.

An anti-slipping adhesive member 140 may be formed along a border between the electrode sensor 110 and the coupling adhesive member 121. The anti-slipping adhesive member 140 may be a hot-melt or silicon-based tape. In this case, even when a user takes vigorous exercise, the electrode sensor 110 coupled to the garment 10 may be stably kept in contact with a skin of the user without slipping owing to the anti-slipping adhesive member 140.

A signal connection line 300 may be connected to the electrode sensor 110 using an interconnection adhesive member 120. For this, an end portion of the signal connection line 300 may be placed on the electrode sensor 110, and then the end portion of the signal connection line 300 may be covered with an interconnection adhesive member 120. The interconnection adhesive member 120 may be a seam sealing or hot-melt tape. The interconnection adhesive member 120 may be more elasticity than the electrode sensor 110. In this case, an electric connection between the electrode sensor 110 and the signal connection line 300 may be stably maintained.

Instead of using the interconnection adhesive member 120, an interconnection metal structure 130 may be used to connect the electrode sensor 110 and the signal connection line 300. For example, the electrode sensor 110 and the signal connection line 300 may be connected using the interconnection metal structure 130 as follows: a hole may be formed through the electrode sensor 110; a metal structure 131 having a post corresponding to the hole may be inserted into the hole; an end portion of the signal connection line 300 may be wound around the post of the metal structure 131; and the metal structure 131 may be deformed into a yoyo shape having a central post and circular disks on both ends of the central post. After the metal structure 131 is deformed, the metal structure 131 may be referred to as an interconnection metal structure 130. Since the signal connection line 300 may be disposed between the electrode sensor 110 and the interconnection metal structure 130, an electric connection between the electrode sensor 110 and the signal connection line 300 may be stably maintained.

In operation S20, finishing may be performed on the signal connection line 300, which is provided to the inner surface of the garment 10 for transmitting physiological signals detected by the electrode sensor 110. For this, a finishing adhesive member 122 may be bonded to the inner surface of the garment 10 with the signal connection line 300 being disposed between the garment 10 and the finishing adhesive member 122. The finishing adhesive member 122 may be a seam sealing or hot-melt tape. The finishing adhesive member 122 may be more elasticity than the signal connection line 300. In this case, even when a user takes vigorous exercise, the finishing adhesive member 122 may stably protect the signal connection line 300.

In operation S30, a snap structure 220 may be formed on a portion of the garment 10 where the electrode sensor 110 is not overlapped and may be connected to the signal connection line 300. The operation S30 may be performed as follows: a portion of the garment 10 where the snap structure 220 is coupled may be selected; a hole may be formed through the selected portion of the garment 10; a male snap 221 having a post corresponding to the hole may be inserted into the hole; an end portion of the signal connection line 300 may be wound around the post; and a female snap 222 may be inserted into an end of the post of the male snap 221 protruding from the outer surface of the garment 10. The snap structure 220 may be formed into a yoyo shape by coupling of the male snap 221 and the female snap 222. The snap structure 220 may include a central post passing through the garment 10, and a pair of circular disks disposed on both ends of the central post. Since the signal connection line 300 is wound around the snap structure 220, an electric connection between the signal connection line 300 and the snap structure 220 may be stably maintained.

A conductive material 230 may be disposed between the garment 10 and the male snap 221. In this case, an electric connection between the male snap 221 and the signal connection line 300 may be more reliable. The snap structure 220 may be finished by attaching a snap structure bonding member 124 to the inner surface of the garment 10 to cover the snap structure 220. The snap structure bonding member 124 may be elastic. The snap structure bonding member 124 may be as elastic as fabric used for making the garment 10 or more elasticity than the fabric. In this case, even when a user takes vigorous exercise, the user's skin may be not injured by the snap structure 220 owing to the snap structure bonding member 124.

In operation S40, a measurement unit is mounted to the snap structure 220. For this, a terminal of the measurement unit may be inserted into a hole of the central post of the snap structure 220. The snap structure 220 and the measurement unit may be connected to each other by an electric connection structure similar to a snap fastening structure.

In operation S50, a pocket unit 400 is formed on an outer surface of the garment 100 to pocket the measurement unit. For this, a sewing or non-sewing method may be used. The pocket unit 400 may be formed of fabric similar to that used for forming the garment 10. According to the non-sewing method, the pocket unit 400 may be attached to the outer surface of the garment 10 using an adhesive.

FIG. 3 is a perspective view illustrating a signal connection line of a physiological signal measuring garment according to an embodiment of the present invention.

Referring to FIG. 3, a signal connection line 300 may include an elastic thread 310, a metal thread 320, and an insulation thread 330. The elastic thread 310 may be a core material, and the metal thread 320 may be wound around the elastic thread 310. The insulation thread 330 may be wound around the metal thread 320 to cover the metal thread 320.

The elastic thread 310 may include an elastic material. The elastic material may be a rubber thread. Therefore, the signal connection line 300 may have elasticity. The metal thread 320 may be coated with a conductive material. The conductive material may include metal such as silver (Ag). Therefore, the signal connection line 300 may be conductive. The insulation thread 330 may include polyester fabric. Therefore, the signal connection line 300 may be protected from external electric interferences.

The elastic thread 310 may be disposed in the core of the signal connection line 300 through various methods, and the metal thread 320 and the insulation thread 330 may be wound through various methods. For example, the diameter, elasticity, and insulating characteristics of the signal connection line 300 may be adjusted according to the elasticity and appearance of the garment 10 (refer to FIG. 1). When the metal thread 320 includes metal such as silver, the signal connection line 300 may have a small diameter and high elasticity.

FIG. 4 is a perspective view illustrating an electrode sensor unit of a physiological signal measuring garment according to an embodiment of the present invention, and FIG. 5 is a sectional view taken along line A-A′ of FIG. 4.

Referring to FIGS. 4 and 5, an electrode sensor unit 100 may include an electrode sensor 110 and an interconnection adhesive member 120.

The electrode sensor unit 100 may be formed as follows: an exposed end portion of a signal connection line 300 from which an insulation thread 330 (refer to FIG. 3) is removed may be placed on the electrode sensor 110 formed of conductive fabric; and the interconnection adhesive member 120 is attached to the electrode sensor 110 to cover the exposed end portion of the signal connection line 300. The end portion of the signal connection line 300 placed on the electrode sensor 110 may have a zigzag shape. In this case, a reliable electrical connection may be formed between the electrode sensor 110 and the signal connection line 300.

The interconnection adhesive member 120 may be a seam sealing or hot-melt tape. The interconnection adhesive member 120 may be more elasticity than the electrode sensor 110. In this case, even when a user takes vigorous exercise, the connection between the electrode sensor 110 and the signal connection line 300 may be stably maintained.

The interconnection adhesive member 120 may be attached to the electrode sensor 110 using a generally-known method. For example, after placing the interconnection adhesive member 120 on the electrode sensor 110, the interconnection adhesive member 120 may be pressed using a roller while applying heat to the interconnection adhesive member 120 using a heat blower.

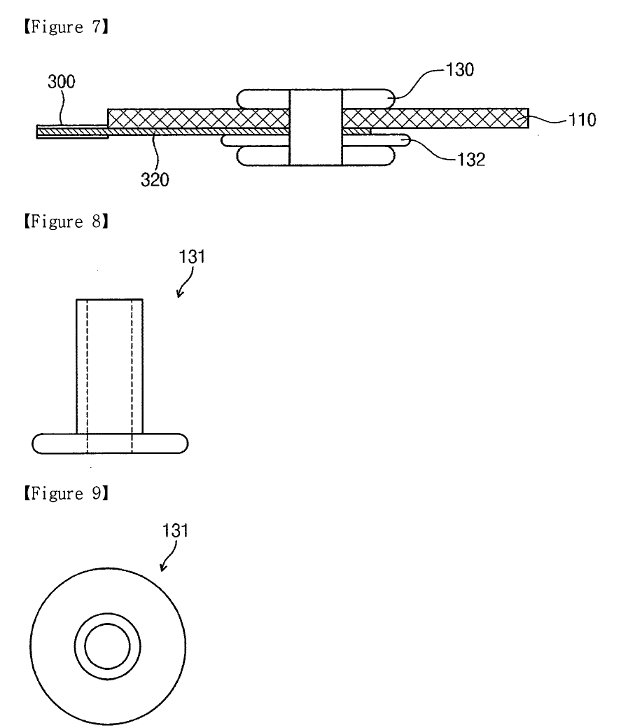

FIG. 6 is a plan view illustrating an electrode sensor unit of a physiological signal measuring garment according to another embodiment of the present invention, and FIG. 7 is a sectional view taken along line B-B′ of FIG. 6. FIGS. 8 and 9 are front and plan views illustrating a metal structure used in the electrode sensor unit of FIG. 6 according to an embodiment of the present invention.

Referring to FIGS. 6 through 9, an electrode sensor unit 100 may include an electrode sensor 110 and an interconnection metal structure 130.

The electrode sensor unit 100 may be formed as follows: a hole may be formed through the electrode sensor 110 formed of conductive fabric; a metal structure 131 having a post corresponding to the hole may be inserted into the hole; an exposed end portion of the signal connection line 300 from which an insulation thread 330 (refer to FIG. 3) is removed may be wound around the post of the metal structure 131; and the metal structure 131 may be deformed into a yoyo shape having a central post and circular disks on both ends of the central post. After the metal structure 131 is deformed, the metal structure 131 may be referred to as the interconnection metal structure 130. An upper portion of the metal structure 131 may be outwardly stretched to form the interconnection metal structure 130. For this, a tool such as a metal rod may be placed on the upper portion of the metal structure 131, and the tool may be beat using a hammer or a punch.

A fixing material 132 may be disposed between the electrode sensor 110 and the interconnection metal structure 130. Owing to the fixing material 132, the signal connection line 300 wound around the central post of the interconnection metal structure 130 may be securely fixed to the interconnection metal structure 130. The fixing material 132 may include plastic or rubber. In this case, a connection between the electrode sensor 110 and the signal connection line 300 may be physically and electrically secured more reliably. Therefore, even when a user takes vigorous exercise, the connection between the electrode sensor 110 and the signal connection line 300 may be stably maintained by the interconnection metal structure 130 and the fixing material 132.

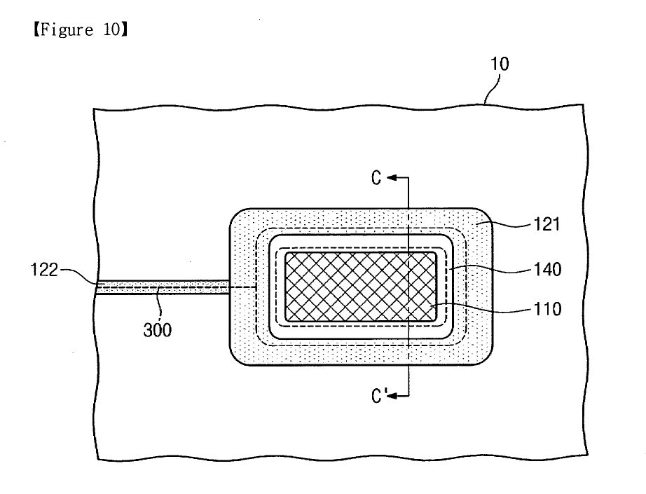

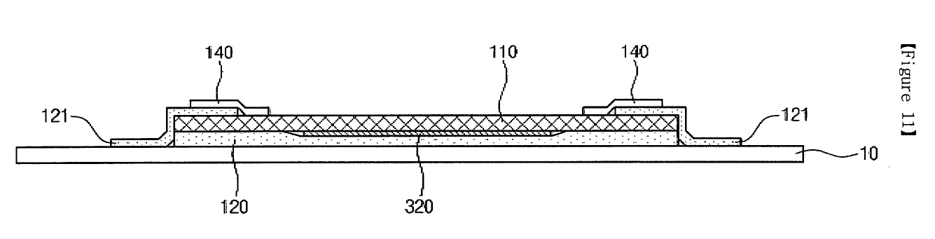

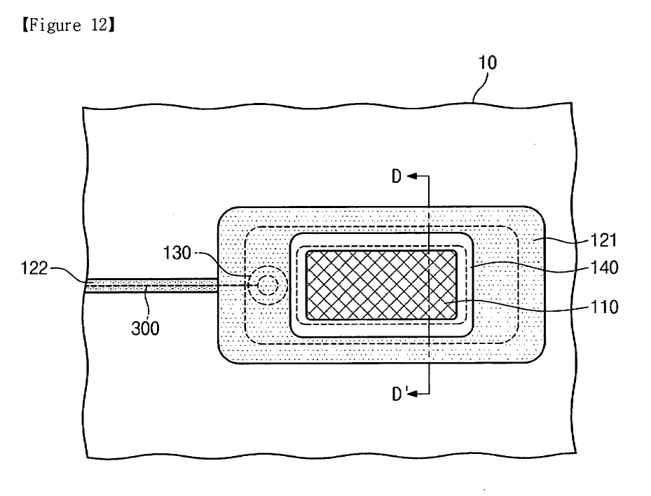

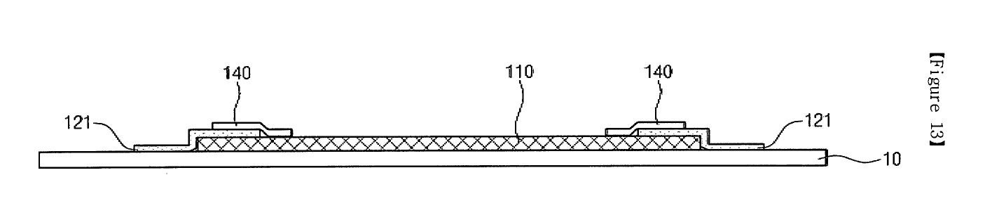

FIGS. 10 and 12 are plan views illustrating coupled electrode sensors and finished signal connection lines to a physiological signal measuring garment according to embodiments of the present invention, and FIGS. 11 and 13 are sectional views taken along lines C-C′ of FIG. 10 and line D-D′ of FIG. 12.

Referring to FIGS. 10 and 11, an electrode sensor unit may include an electrode sensor 110 and a signal connection line 300 that are electrically connected using an interconnection adhesive member 120. The electrode sensor unit may be coupled to a desired portion of a garment 10 using a coupling adhesive member 121.

The coupling adhesive member 121 may have an opened frame shape for attaching edges of the electrode sensor 110 to the garment 10. In this case, a center portion of the electrode sensor 110 may be exposed for making contact with a user's skin. If the electrode sensor 110 has a rectangular plate shape, the coupling adhesive member 121 may have an opened rectangular frame shape. If the electrode sensor 110 has a circular plate shape, the coupling adhesive member 121 may be ring-shaped. The coupling adhesive member 121 may be formed by bonding a piece of fabric used for making the garment 10 to a seam sealing or hot-melt tape. For example, the coupling adhesive member 121 may be formed by bonding a piece of fabric used for making the garment 10 to a hot-melt tape. In this case, it may be difficult to distinguish the coupling adhesive member 121 from the garment 10, and thus the garment 10 may have a neat appearance.

An anti-slipping adhesive member 140 may be formed along a border between the electrode sensor 110 and the coupling adhesive member 121. In this case, even when a user takes vigorous exercise, the electrode sensor 110 coupled to the garment 10 may be stably kept in contact with the skin of the user without slipping owing to the anti-slipping adhesive member 140. The anti-slipping adhesive member 140 may be a hot-melt or silicon-based tape.

A portion of the signal connection line 300 that is not placed on the electrode sensor 110 may be attached to an inner surface of the garment 10 using a finishing adhesive member 122. The finishing adhesive member 122 may be more elasticity than the signal connection line 300. In this case, even when a user takes vigorous exercise, the signal connection line 300 may be stably protected owing to the finishing adhesive member 122.

The signal connection line 300 may be finished with the finishing adhesive member 122 using a generally-known method. For example, after placing the finishing adhesive member 122 on the garment 10 to cover the signal connection line 300, the finishing adhesive member 122 may be pressed using a roller while applying heat to the finishing adhesive member 122 using a heat blower.

Referring to FIGS. 12 and 13, an electrode sensor unit may include an electrode sensor 110 and a signal connection line 300 that are electrically connected using an interconnection metal structure 130. The electrode sensor unit may be coupled to a desired portion of a garment 10 using a coupling adhesive member 121.

The coupling adhesive member 121 may have an opened frame shape for attaching edges of the electrode sensor 110 to the garment 10. In this case, a center portion of the electrode sensor 110 may be exposed for making contact with a user's skin. If the electrode sensor 110 has a rectangular plate shape, the coupling adhesive member 121 may have an opened rectangular frame shape. If the electrode sensor 110 has a circular plate shape, the coupling adhesive member 121 may be ring-shaped. The coupling adhesive member 121 may be formed by bonding a piece of fabric used for making the garment 10 to a seam sealing or hot-melt tape. For example, the coupling adhesive member 121 may be formed by bonding a piece of fabric used for making the garment 10 to a hot-melt tape. In this case, it may be difficult to distinguish the coupling adhesive member 121 from the garment 10, and thus the garment 10 may have a neat appearance.

An anti-slipping adhesive member 140 may be formed along a border between the electrode sensor 110 and the coupling adhesive member 121. In this case, even when a user takes vigorous exercise, the electrode sensor 110 coupled to the garment 10 may be stably kept in contact with the skin of the user without slipping owing to the anti-slipping adhesive member 140. The anti-slipping adhesive member 140 may be a hot-melt or silicon-based tape.

A portion of the signal connection line 300 that is not placed on the electrode sensor 110 may be attached to an inner surface of the garment 10 using a finishing adhesive member 122. The finishing adhesive member 122 may be more elasticity than the signal connection line 300. In this case, even when a user takes vigorous exercise, the signal connection line 300 may be stably protected owing to the finishing adhesive member 122.

The signal connection line 300 may be finished with the finishing adhesive member 122 using a generally-known method. For example, after placing the finishing adhesive member 122 on the garment 10 to cover the signal connection line 300, the finishing adhesive member 122 may be pressed using a roller while applying heat to the finishing adhesive member 122 using a heat blower.

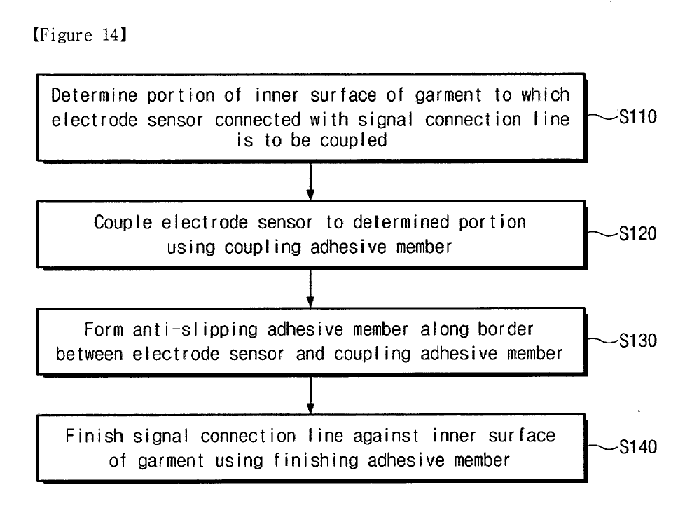

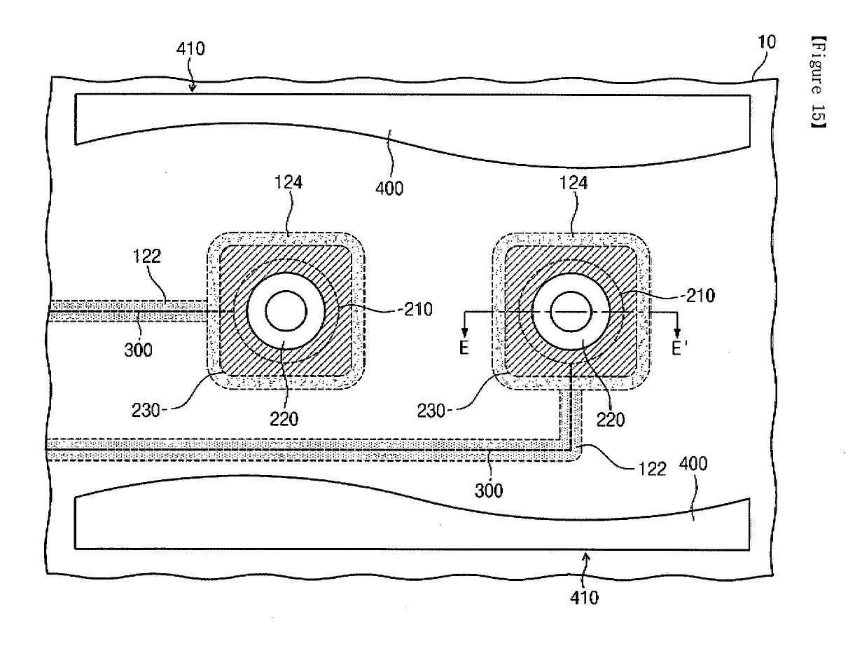

FIG. 14 is a flowchart for explaining a method of coupling an electrode sensor to a physiological signal measuring garment and finishing a signal connection line according to an embodiment of the present invention. The elements shown in FIGS. 10 and 11 will be used for explaining the method.

Referring to FIG. 14, according to the method of the current embodiment, an electrode sensor 110 may be attached to a physiological signal measuring garment and a signal connection line 300 may be finished as follows. In operation S110, a portion of an inner surface of a garment 10 may be selected to attach the electrode sensor 110 connected with the signal connection line 300 to the selected portion of the inner surface of the garment 10. In operation S120, the electrode sensor 110 may be attached to the selected portion of the inner portion of the garment 10 using a coupling adhesive member 121. In operation S130, an anti-slipping adhesive member 140 may be formed along a border between the electrode sensor 110 and the coupling adhesive member 121. In operation S140, the signal connection line 300 may be attached to an inner surface of the garment 10 using a finishing adhesive member 122.

In operation S110, the portion of the inner surface of the garment 10 where the electrode sensor 110 to be attached may be selected according to the kind of physiological signals to be measured as explained in FIG. 1. In operation S120, the electrode sensor 110 may be attached to the selected portion of the inner surface of the garment 10 using the coupling adhesive member 121 as described above. In operation S130, the anti-slipping adhesive member 140 may be formed for stably keeping the electrode sensor 110 in contact with a user's skin without slipping. In operation S140, the signal connection line 300 may be attached to the inner surface of the garment 10 using the finishing adhesive member 122 so as to securely protect the signal connection line 300 even when a user takes vigorous exercise.

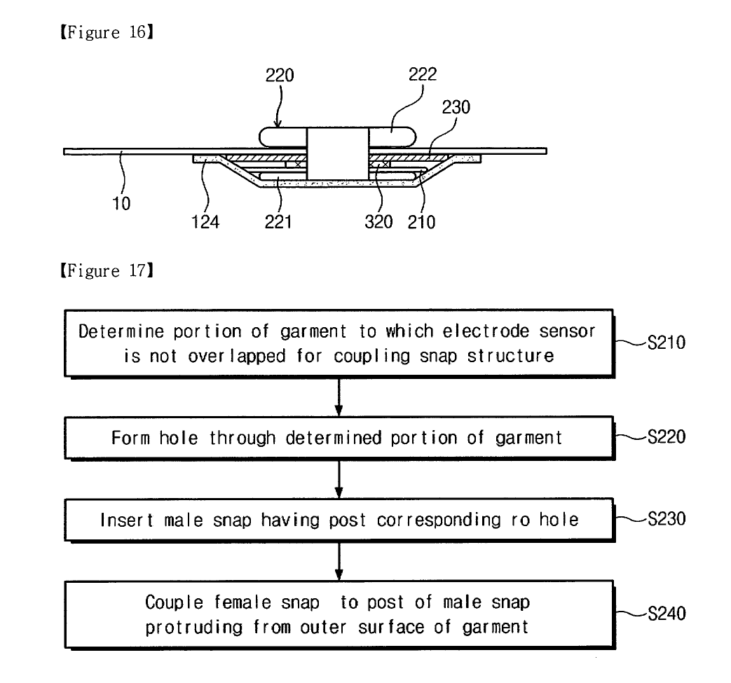

FIG. 15 is a plan view illustrating snap structures coupled to a physiological signal measuring garment according to an embodiment of the present invention, and FIG. 16 is a sectional view taken along line E-E′ of FIG. 15.

Referring to FIGS. 15 and 16, a snap structure 220 (two are shown) electrically connected with a signal connection line 300 may be coupled to any portion of a garment 10 where an electrode sensor unit 100 (refer to FIG. 1) is not overlapped. For this, a snap structure bonding member 124 may be used.

The snap structure 220 may be formed as follows: a hole may be formed through the garment 10; a male snap 221 having a post corresponding to the hole may be inserted into the hole; an end portion of the signal connection line 300 from which an insulation thread 330 (refer to FIG. 3) is removed may be wound around the post; and a female snap 222 may be coupled to an end of the post of the male snap 221 protruding from the outer surface of the garment 10. The snap structure 220 may be formed into a yoyo shape by coupling of the male snap 221 and the female snap 222. The snap structure 220 may include a central post passing through the garment 10, and a pair of circular disks disposed on both ends of the central post. Since the signal connection line 300 is wound around the snap structure 220, an electric connection between the signal connection line 300 and the snap structure 220 may be stably maintained. The snap structure 220 may formed by coupling the female snap 222 to the male snap 221. This configuration of the snap structure 220 may be selected according to the design and the structure of a measurement unit (not shown).

A snap fixing material 210 may be disposed between the garment 10 and the snap structure 220. The snap fixing material 210 may include plastic or rubber. Owing to the snap fixing material 210, the signal connection line 300 wound around the central post of the snap structure 220 may be securely fixed. Therefore, physical and electrical connection between the snap structure 220 and the signal connection line 300 may be more reliable. Accordingly, a connection between the snap structure 220 and the signal connection line 300 may be securely kept owing to the snap fixing material 210 even when a user takes vigorous exercise.

A conductive material 230 may be disposed between the garment 10 and the male snap 221. The conductive material 230 may include a conductive material for transmitting electric signals. The conductive material may include conductive fabric or conductive rubber. In this case, an electric connection between the snap structure 220 and the signal connection line 300 may be more reliable.

The snap structure bonding member 124 may be attached to an inner surface of the garment 10 to cover the snap structure 220. In this case, even when a user takes vigorous exercise, the user's skin may be protected from the snap structure 220. The snap structure bonding member 124 may be formed by bonding a piece of fabric used for making the garment 10 to a seam sealing or hot-melt tape. For example, the snap structure bonding member 124 may be formed by bonding a piece of fabric used for making the garment 10 to a hot-melt tape. In this case, it may be difficult to distinguish the snap structure bonding member 124 from the garment 10, and thus the garment 10 may have a neat appearance. A portion of the signal connection line 300 not placed on the snap structure 220 may be attached to the inner surface of the garment 10 using a finishing adhesive member 122.

Reference numeral 400 denotes a pocket unit, and reference numeral 410 denotes a boundary of the pocket unit 400. The pocket unit 400 may provide a room for pocketing a measurement unit (not shown) to be mounted to the snap structure 220. The pocket unit 400 may cover the snap structure 220. In FIG. 15, a portion of the pocket unit 400 is cut away to show the snap structure 220 coupled to the garment 10. The boundary 410 may show a portion of the garment 10 where the pocket unit 400 is formed. The pocket unit 400 may be attached to the garment 10 by a sewing or non-sewing method. The pocket unit 400 may be formed of fabric similar to that used for making the garment 10. According to the non-sewing method, the pocket unit 400 may be attached to an outer surface of the garment 10 using an adhesive.

FIG. 17 is a flowchart for explaining a method of forming a snap structure of a physiological signal measuring garment according to an embodiment of the present invention. The method will now be described with reference to the elements shown in FIGS. 15 and 16.

Referring to FIG. 17, a snap structure 220 may be formed as follows. In operation S210, a portion of a garment 10 where an electrode sensor is not overlapped may be selected so as to couple the snap structure 220 to the selected portion of the garment 10. In operation S220, a hole may be formed through the selected portion of the garment 10. In operation S230, a male snap 221 having a post corresponding to the hole may be inserted into the hole. In operation S240, a female snap 222 may be coupled to an end portion of the male snap 221 protruding from an outer surface of the garment 10.

In operation S210, any portion of the garment 10 where an electrode sensor is not overlapped may be selected according to the design, convenience, and purpose of the garment 10 as described in FIG. 1. Then, the snap structure 220 may be formed through operations S220, S230, and S240. A measurement unit may be mounted to the snap structure 220.

According to the embodiments of the present invention, the garment, the electrode sensor, and the signal connection line of the physiological signal measuring garment are elastic. Therefore, although the physiological signal measuring garment can be folded and/or stretched when a user moves or takes exercise, distortion and noise of detected physiological signals can be kept below a low level. That is, the present invention can provide a physiological signal measuring garment for easily detecting physiological signals even when a user moves or takes vigorous exercise, and a method of fabricating the physiological signal measuring garment. Furthermore, according to the physiological signal measuring garment and the method of fabricating the same, since an electrode sensor can be attached to a desired portion of a garment, various physiological signals can be detected.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

1-51. (canceled)

52. A garment for measuring physiological signals, comprising:
an electrode sensor coupled to an inner surface of a garment to make contact with a skin for detecting physiological signals;
a signal connection line connected to the electrode sensor;
a snap structure electrically connected to the signal connection line; and
a measurement unit mounted on the snap structure for measuring the physiological signals,
wherein the signal connection line has elasticity.

53. The garment of claim 52, wherein a portion of the garment which coupled to the electrode sensor is designed for applying a pressure equal to or higher than 0.1 kPa.

54. The garment of claim 52, wherein the electrode sensor is a conductive fabric electrode formed of conductive yarn, the conductive fabric electrode having a stronger elasticity than the garment.

55. The garment of claim 52, further comprising a coupling adhesive member used to couple the electrode sensor to the inner surface of the garment.

56. The garment of claim 52, further comprising an interconnection adhesive member configured to connect the electrode sensor and the signal connection line, the interconnection adhesive member having a stronger elasticity than the electrode sensor.

57. The garment of claim 52, further comprising an interconnection metal structure configured to connect the electrode sensor and the signal connection line.

58. The garment of claim 52, wherein the signal connection line comprises:
an elastic thread as a core material:
a metal thread wound around the elastic thread; and
an insulation thread wound around the metal thread to cover the metal thread.

59. The garment of claim 52, wherein the signal connection line is finished against the inner surface of the garment.

60. The garment of claim 52, wherein the snap structure comprises:
a male snap comprising a post passing through the garment; and
a female snap coupled to the male snap with the garment being disposed between the female snap and the male snap.

61. The garment of claim 52, wherein the snap structure is coupled to a portion of the garment to which the electrode sensor is not overlapped.

62. A method of fabricating a physiological signal measuring garment, the method comprising:
coupling an electrode sensor to an inner surface of a garment to allow the electrode sensor to make contact with a skin for detecting physiological signals;
connecting a signal connection line to the electrode sensor;
forming a snap structure electrically connected to the signal connection line; and
mounting a measurement unit on the snap structure,
wherein the signal connection line has elasticity.

63. The method of claim 62, wherein a portion of the garment which coupled to the electrode sensor is designed for applying a pressure equal to or higher than 0.1 kPa.

64. The method of claim 62, wherein the electrode sensor is a conductive fabric electrode formed of conductive yarn by a tricot method or a knit method.

65. The method of claim 62, wherein the coupling of the electrode sensor to the inner surface of the garment comprises:
determining a portion of the garment to which the electrode sensor is to be coupled; and
coupling the electrode sensor to the determined portion of the garment using a coupling adhesive member.

66. The method of claim 62, wherein the connecting the signal connection line to the electrode sensor uses an interconnection adhesive member.

67. The method of claim 62, wherein the connecting the signal connection line to the electrode sensor uses an interconnection metal structure.

68. The method of claim 62, wherein the signal line is formed by a method comprising:
preparing an elastic thread using a core material having an elastic material;
winding the elastic thread with a metal thread coated with a conductive material; and
winding the metal thread with an insulation thread formed of polyester fabric to cover the metal thread.

69. The method of claim 62, further comprising finishing the signal connection line against the inner surface of the garment.

70. The method of claim 62, wherein the forming of the snap structure comprises:
determining a portion of the garment to which the snap structure is coupled;
forming a hole through the determined portion of the garment;
inserting a male snap having a post corresponding to the hole into the hole; and
coupling a female snap to a portion of the post of the male snap protruding from an outer surface of the garment so as to form the snap structure into a yoyo shape in which a pair of circular disks are disposed on both sides of a central post passing through the garment.

71. The method of claim 62, wherein the snap structure is coupled to a portion of the garment to which the electrode sensor is not overlapped.
